(19) United States
(12) Patent Application Publication
(10) Pub. No.: US 2008/0152736 A1
(43) Pub. Date: Jun. 26, 2008

(54) Lipid Fraction of Nigella Sativa L. Seeds

(76) Inventor: Osama Kandil, Cairo (EG)

Correspondence Address: SMITH PATENT CONSULTING CONSULTING, LLC, 3309 DUKE STREET, ALEXANDRIA, VA, 22314, US

(21) Appl. No.: 12/043,052

(22) Filed: Mar. 5, 2008

Related U.S. Application Data
(62) Division of application No. 10/809,856, filed on Mar. 26, 2004.

(51) Int. Cl. A61K 36/28

(52) U.S. Cl. 424/764

(57) ABSTRACT

The present invention provides novel compositions comprising a Nigella sativa L. lipid fraction. The Nigella sativa L. lipid fraction is an oily fraction primarily comprised of long chain fatty acids, volatile oils and sterols. The present invention also provides novel methods using compositions comprising the Nigella sativa L. lipid fraction for treating and preventing skin infections, wounds, bacterial infections, respiratory diseases and disorders, cellulite, cardiovascular disorders, septic infections, and the like.

### FIELD OF THE INVENTION

The present invention is generally directed to the field of medicine and pharmacology, particularly to compounds and compositions extracted from Nigella sativa L. seeds, and to methods of using such compounds and compositions. More particularly, the present invention is directed to a lipid fraction extracted from Nigella sativa L. seeds and novel medicinal uses thereof.

### BACKGROUND OF THE INVENTION

A variety of herbal and plant extracts are available today for treating any number of diseases affecting the human body. Some preparations have been known for thousands of years while others are just being discovered to have highly curative effects. Effective plant extracts are highly desired as a natural way to treat diseases. It is believed that natural preparations will not have an adverse effect on the body compared to synthetic preparations.

The Nigella sativa plant is cultivated for its seeds which are used for medicinal purposes. Nigella sativa L seeds come from an herbaceous plant that belongs to the Ranunculaceae family. The plant is a dicotyledon and is cultivated in various parts of the world, especially in Eastern Mediterranean countries and also in India, Bangladesh, Turkey and Pakistan. It is also grown in other places having similar climates, such as East Africa and Middle Europe. The plant is characterized by an erect branched stem and alternating, finely divided, feathery, grayish-green leaves. The bluish-white, star-shaped flowers are terminal and solitary, and there are no petals. The fruit is a globase capsule with small black, rough seeds. Other species of Nigella include Nigella arvensis and Nigella damascena.

The seeds of Nigella sativa are known as Habbatul Baraka (meaning the seed of good fortune) and El Habba El Sawdaa (meaning Black Seed) in the Mediterranean region. The seeds are also known as Kalajira or Kalaoji and Black Cumin in the Indian subcontinent. Nigella sativa L. seeds have been used as a natural remedy for over 4,000 years in various parts of the world, particularly in the Near and Middle East. In the authentic tradition and sayings of the Prophet Muhammad (peace be upon him) which are documented in “Hadeeth” books he is quoted as saying: “In the Black Seed there is healing for every illness except death.”

There is a need in the art for new pharmaceutical compounds and compositions that are derived from natural sources, like plants. The present invention is directed to this, as well as other, important ends.

### SUMMARY OF THE INVENTION

The present invention provides novel compositions comprising a Nigella sativa L. lipid fraction. The Nigella sativa L. lipid fraction is an oily fraction comprised of polyunsaturated fatty acids, saturated fatty acids, glyceryl esters, volatile oils and sterols. The compositions are preferably formulated for topical administration and preferably contain a pharmaceutically acceptable carrier.

In one embodiment, the present invention provides a topical composition comprising a Nigella sativa L. lipid fraction and a pharmaceutically acceptable carrier. The lipid fraction is extracted from the seeds of Nigella sativa L in a manner that yields an oily fraction that is comprised primarily of long chain fatty acids, volatile oils and sterols and that is free from Nigella sativa L solid fats, including, for example, waxes, resins, tocopherols, triterpenes, aglycons, short chain fatty acids, and hydrocarbons. In a preferred embodiment, the lipid fraction is present in an amount ranging from about 1 to about 20% by weight based on 100% by weight of the total composition.

The topical composition of the present invention may be formulated for topical administration as an ointment, cream, gel, powder, balm, lotion, liquid spray or aerosol, for example, or as the active ingredient of a transdermal patch.

In a further embodiment, the present invention provides novel medicinal uses for the Nigella sativa L. lipid fraction, including, but not limited to, methods for treating a pyogenic skin infection, methods for treating skin, soft tissue, septic and/or bacterial infections, methods for healing wounds, methods for treating cellulite, and methods for treating or preventing vaginal diseases or disorders, methods for treating respiratory diseases or disorders, comprising the step of topically administering an effective amount of a topical composition comprising a Nigella sativa L. lipid fraction, preferably in combination with a pharmaceutically acceptable carrier.

These and other objects of the invention will be evident from the following description, taken together with the attached drawings and appended claims. It is to be understood that both the foregoing summary of the invention and the following detailed description are of a preferred embodiment, and not restrictive of the invention or other alternate embodiments of the invention.

### BRIEF DESCRIPTION OF THE FIGURE

FIG. 1 shows the process for preparing the Nigella sativa L. lipid fraction of the present invention from Nigella sativa L. seeds.

FIG. 2 shows the percent concentration of components present in Nigella sativa L. seeds after the various treatments and extractions described in FIG. 1.

### DETAILED DESCRIPTION OF THE INVENTION

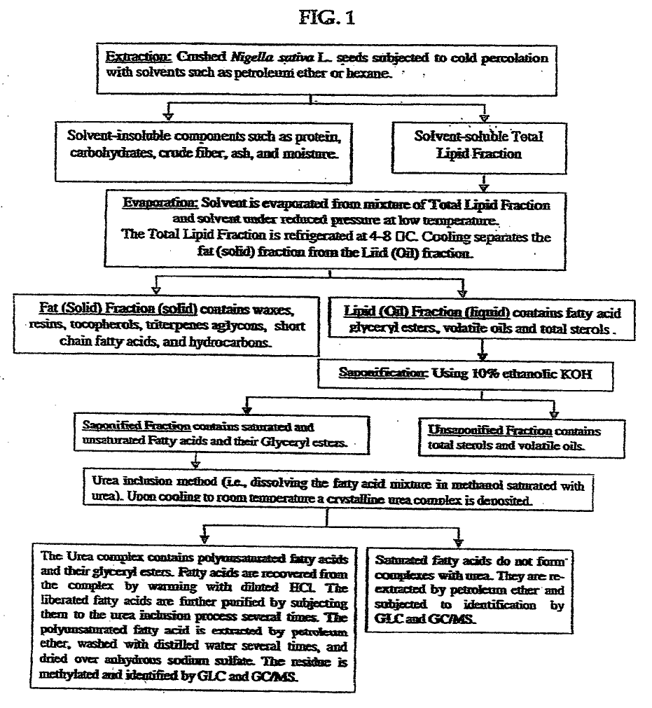

The present invention describes novel compositions comprising the lipid fraction from Nigella sativa L. seeds. The “Nigella sativa L. lipid fraction” refers to the lipid fraction extracted from the seeds of Nigella sativa L. The “Nigella sativa L. lipid fraction” can be extracted from Nigella sativa L. seeds following the methods described in Example 1 and FIG. 1. The lipid fraction extracted from the seeds of Nigella sativa L comprises the polyunsaturated fatty acids described herein, the saturated fatty acids described herein, the volatile oils described herein, the total sterols described herein and the other components described herein.

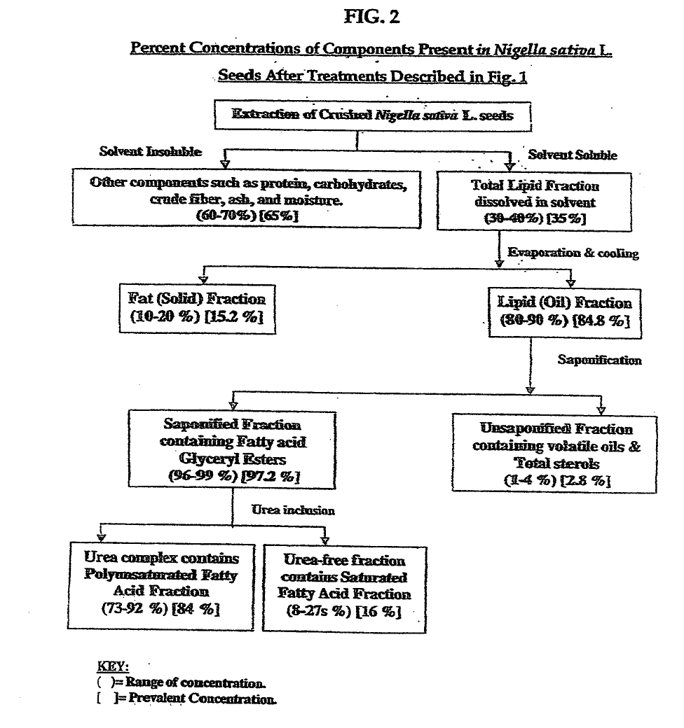

The Nigella sativa L. lipid fraction results from evaporating and cooling the solvent-extracted total lipid fraction as described in Example 1 and FIGS. 1 and 2. This lipid fraction is present in an amount of about 80 to about 90% (more preferably about 84.8%) of the total lipid fraction. The remaining 10-20% (more preferably about 15.2%) is a solid (fat) fraction. The lipid fraction contains, in addition to the fatty acid fraction described below, volatile oils in an amount of about 0.1 to about 1% (more preferably about 0.5%), and total sterols in an amount of about 1 to about 3% (more preferably about 2.3%). Volatile oils and total sterols make up about 1 to about 4% (more preferably about 2.8%) of the lipid fraction. The remaining 96-99% (more preferably about 97.2%) constitutes the total fatty acid fraction containing fatty acid glyceryl esters as described below.

The polyunsaturated fatty acid fraction is present in an amount of about 73 to about 92% by weight (preferably about 84% by weight) in the total fatty acid fraction. The saturated fatty acid fraction is present in an amount of about 8 to about 27% by weight (preferably about 16% by weight) of the total fatty acid fraction.

In the total fatty acid fraction, the Nigella sativa L. polyunsaturated fatty acid fraction comprises about 51 to about 61% by weight octadecadienoic acid (i.e., substantially in the form of cis-9,12-octadecadienoic acid (i.e., linoleic acid)); about 20 to about 25% by weight octadecenoic acid (i.e., substantially in the form of cis-9-octadecenoic acid (i.e., oleic acid)); about 0.7 to about 2% by weight cis-9,12,15-octadecatrienoic acid (i.e., linolenic acid); cis-11,14-eicosadienoic acid (preferably in an amount of about 1 to about 2.6% by weight); cis-9-tetradecenoic (i.e., myristoleic acid) (preferably in an amount of about 0.10 to about 0.21% by weight); and cis-9-hexadecenoic (i.e., palmitoleic acid) (preferably in an amount of about 0.30 to about 1.0% by weight), as shown in Table 1 below. One skilled in the art will recognize that the compounds described herein may exist in more than one isomeric and/or derivative form, such as, for example, glyceryl esters.

In the total fatty acid fraction, the saturated fatty acid fraction comprises about 11 to about 14% N-hexadecanoic acid (i.e., palmitic acid); about 0.1 to about 1% tetradecanoic acid (i.e., myristic acid); about 0.14 to about 3% eicosanoic acid (i.e., arachidic acid); about 0.5 to about 3.2% octadecanoic acid (i.e., stearic acid); and about 0.8 to about 1.3% tetracosanoic acid (i.e., lignoseric acid), as shown in Table 2 below. One skilled in the art will recognize that the compounds described herein may exist in more than one isomeric and/or derivative form, such as, for example, a glyceryl ester.

TABLE 1
Polyunsaturated Fatty Acid Fraction

| | | | % by weight in | Abbreviated |
|---|---|---|---|---|
| No. | IUPAC Name | Common | fraction | Structure |
| 1. | cis-9,12-octadecadienoic acid | Linoleic acid | 60.7-72.6% | C18:2 |
| 2. | cis-9-octadecenoic acid | Oleic acid | 23.8-29.7% | C18:1 |
| 3. | cis-11,14-eicosadienoic acid | | 1.2-3.1% | C20:2 |
| 4. | cis-9,12,15-octadecatrienoic | Linolenic acid | 0.83-2.38% | C18:3 |
| | acid | | | |
| 5. | cis-9-hexadecenoic acid | Palmitoleic acid | 0.36-1.2% | C16:1 |
| 6. | cis-9-tetradecenoic acid | Myristoleic acid | 0.12-0.25% | C14:1 |

TABLE 2
Saturated Fatty Acid Fraction

| | | | % by Weight in | Abbreviated |
|---|---|---|---|---|
| No. | IUPAC Name | Common Name | Fraction | Structure |
| 1. | Hexadecanoic | Palmitic | 11-14% | C16:0 |
| 2. | Octadecanoic | Stearic | 0.5-3.2% | C18:0 |
| 3. | Eicosanoic | Arachidic | 0.14-3% | C20:0 |
| 4. | Tetracosanoic | Lignoseric | 0.8-1.3% | C24:0 |
| 5. | Tetradecanoic | Myristic | 0.1-1% | C14:0 |

The volatile oils contain about 24% thymoquinone and minor amounts of other volatile materials, such as terpene hydrocarbons (such as ∀-pinene, ∃-pinene, Δ-cymene, camphene, terpinene) and other oxygenated volatile oils (such as ∀-terpinol, linalool and borneol). A GC/MS analysis of the volatile oil components of Nigella sativa L. seeds indicates that the volatile oil contains about 46% monoterpenes, about 25% carbonyl compounds, about 24% thymoquinone and dihydrothymoquinone, and 29% other constituents (e.g., alcohols, esters and phenols).

The sterol component comprises ∃-sitosterol, Beta-amyrin, stigmasterol and campesterol.

The “other components” in the Nigella sativa L. total lipid fraction include phenolic compounds such as tocopherols, triterpenes aglycon, short chain fatty acids, hydrocarbons, wax and resins.

In preferred embodiments, the present invention describes novel compositions comprising the Nigella sativa L. lipid fraction of the present invention in an amount of about 1 to about 20% by weight, based on 100% by weight of the composition, preferably in an amount of about 2 to about 15% by weight, more preferably in an amount of about 4 to about 12% by weight, most preferably in an amount of about 5 to about 10% by weight.

The present invention provides novel methods for treating wounds (including infected wounds) in a patient in need thereof by administering, preferably topically administering, an effective amount of a composition comprising the Nigella sativa L. lipid fraction of the present invention. The method for treating wounds is preferably a method for healing wounds. Preferably the composition is administered topically. The wound can be, for example, an infected cut or burn, and is preferably a diabetic leg ulcer. The compositions of the present invention are considered potent immunomodulators with important implications for the treatment and restoration of immune dysfunctions.

The present invention also provides methods for treating skin and soft tissue infections caused by gram-positive organisms or gram-negative bacilli in a patient in need thereof by administering, preferably topically administering, an effective amount of a composition comprising the Nigella sativa L. lipid fraction of the present invention. Exemplary gram-positive organisms include Staphylococcus aureus, Streptococcus pyogenes, and the like. Although gram-negative bacilli infections are rare, they do occur in patients who are diabetic or immunocompromised (e.g., patients with AIDS, HIV infection or transplants).

The present invention provides novel methods for modulating bacterial growth by administering, preferably topically administering, an effective amount of a composition comprising the Nigella sativa L. lipid fraction of the present invention. The growth of any bacteria known in the art can be modulated with the compositions of the present invention. Exemplary bacteria whose growth can be modulated include, but are not limited to, those from the genus Staphylococcus (e.g., Staphyloccus aureus, staphyloccus pyogenes), Corynebacterium (e.g., Corynebacterium pyogenes), Streptococcus (e.g., Streptococcus pyogenes), Salmonella (e.g., Salmonella typhi murium), Escherichia (e.g., Escherichia coli), Pseudomonas (e.g., Pseudomonas aeroginosa), and Kiebsiella (e.g., Klebsiella spp.).

“Modulating bacterial growth” refers to killing or eliminating the bacteria, reducing the amount of bacteria (e.g., compared to the amount of bacteria present before the composition of the present invention was applied), or slowing the rate of growth of the bacteria (e.g., compared to the rate of growth of the bacteria absent the presence of the composition of the present invention).

The present invention also provides novel methods for treating and preventing bacterial infections in a patient in need thereof by administering, preferably topically administering, an effective amount of a composition comprising the Nigella sativa L. lipid fraction of the present invention. Any bacterial infection known in the art can be treated or prevented with the compositions of the present invention. Exemplary bacterial infections that can be prevented or treated with the compositions of the present invention include, but are not limited to, those from the genus Staphylococcus (e.g., Staphylococcus aureus, staphyloccus pyogenes), Corynebacterium (e.g., Corynebacterium pyogenes), Streptococcus (e.g., Streptococcus pyogenes), Salmonella (e.g., Salmonella typhi murium), Escherichia (e.g., Escherichia coli), Pseudomonas (e.g., Pseudomonas aeroginosa), and Klebsiella (e.g., Klebsiella spp.).

“Treating and preventing bacterial infections” includes eliminating or curing the bacterial infection, reducing the severity of the bacterial infection (e.g., compared to the severity of the bacterial infection before the compositions of the present invention were administered), and/or reducing the rate of growth of the bacterial infection (e.g., compared to the rate of growth of the fungal infection in the absence of the compositions of the present invention). The term “patient” refers to animals, preferably mammals, more preferably humans, and includes infants, children and adults.

The present invention provides methods for treating pyogenic skin infections in a patient in need thereof by administering, preferably topically administering, an effective amount of a composition comprising the Nigella sativa L. lipid fraction of the present invention. “Pyogenic skin infections” include, for example, pyoderma, impetigo, folliculitis, eczema (including infected eczema), infected wounds, insect bites, leg ulcers (including diabetic leg ulcers), dermal injuries, erythema, and the like.

Impetigo is a disease caused by superficial bacterial infections of the skin. Such bacterial infections are caused by streptococci or Staphylococcus aureus. Lesions caused by streptococci are characterized by primary lesions in the skin that rupture form a characteristic yellow-brown “honey-colored” crust. Lesion caused by Staphylococci may be in the form of clear bullae and this less common form of the disease is called bullous impetigo.

The present invention also provides novel methods for treating cellulite in a patient in need thereof by administering, preferably topically administering, an effective amount of a composition comprising the Nigella sativa L. lipid fraction of the present invention. Cellulite is an acute inflammatory condition of the skin that is characterized by pain, erythema, swelling and heat and is caused by gram-negative bacilli, including P. aeruginosa which is most common in hospitalized, immunocompromised patients (e.g., patients with AIDS, HIV infection or transplants).

The present invention provides a method for regulating and normalizing certain cardiovascular parameters, such as lowering LDL cholesterol, in a patient in need thereof by administering an effective amount of a composition comprising the Nigella sativa L. lipid fraction of the present invention. The composition comprising the Nigella sativa L. lipid fraction can optionally be administered in conjunction with common antibiotics, such as ampicillin or gentamycin. Recent research indicates that the health-promoting benefits of a plant-based diet may be due to the presence of plant-derived cholesterol analogs such as sterols and sterolins. These compounds, which are structurally similar to cholesterol, appear to have important immunomodulatory and anti-inflammatory activities in human and animal physiology. Human research indicates that plant sterols and sterolins have important anti-inflammatory, anti-ulcer, anti-diabetic, anti-cancer, and T-cell proliferative activities. Medical uses already include the treatment of hyper-cholesterolemia, benign prostatic hypertrophy and rheumatoid arthritis. Plant sterols and sterolins are thought to be responsible for the health benefits of a variety of medicinal herbs including saw palmetto, pygeum, pumpkin seeds and Nigella sativa L. Page 1, South African Journal of Science, 93:263-268 (June 1997).

Although plant sterols (phytosterols) and cholesterol have similar chemical structures, they differ markedly in their synthesis, intestinal absorption and metabolic fate. Phytosterols inhibit intestinal cholesterol absorption, thereby lowering plasma total and low-density lipoprotein (LDL) cholesterol levels. In 16 recently published human studies that used phytosterols to reduce plasma cholesterol levels in a total of 590 subjects, phytosterol therapy was accompanied by an average 10% reduction in total cholesterol and a 13% reduction in LDL cholesterol levels. Moghadasian et al, Am. J. Med., 107(6):588-594 (1999).

The present invention also provides an effective and affordable therapy, with no side-effects, for treating infections through improving immune system function by either balancing an overactive immune system or by enhancing an immune system suppressed by illness or stress (e.g., as observed in infections and cancer) in a patient in need thereof by administering, preferably topically administering, an effective amount of a composition comprising the Nigella sativa L. lipid fraction of the present invention. The compositions of the present invention promote secretion of immune factors, which, in turn help fight off viral and bacterial invaders.

The present invention provides a composition for treatment of nipple fissures in a patient in need thereof by administering, preferably topically administering, an effective amount of a composition comprising the Nigella sativa L. lipid fraction of the present invention.

The present invention also provides methods for treating septic infections in a patient in need thereof by administering, preferably topically administering, an effective amount of a composition comprising the Nigella sativa L. lipid fraction of the present invention.

The present invention provides methods for treating and preventing respiratory diseases and disorders by administering, preferably topically administering, an effective amount of a composition comprising the Nigella sativa L. lipid fraction of the present invention. “Respiratory diseases and disorders” includes, for example, bronchospasms, bronchoconstriction, anaphylaxis, pulmonary tuberculosis, inflamed lung tissues, inflamed mucous membranes, asthma (including bronchial asthma and seasonal asthma), respiratory tract inflammations, bronchitis (e.g., due to inhalation of allergenic substances or fine dust), bronchial hyperactivity, dry cough, bronchial congestion, minor throat irritations, upper respiratory allergies (including those associated with sinusitis as a result of a common cold or the inhalation of irritants), and the like.

While not wishing to be bound by theory, it is believed that the compositions of the invention are useful in treating respiratory diseases and disorders because they produce anti-inflammatory eicosanoids (e.g. PGE); have mast cell stabilizing effects; and have inhibitory effects on the release of histamine, serotonin, and other inflammatory mediators.

With respect to the treatment of pulmonary tuberculosis, for example, the compositions of the present invention appear to increase lymphocytes and eosinophils. The increase in eosinophils may be due to the relationship between the rise of CD4 lymphocytes and eosinophils. Thus, the compositions of the invention may have a positive role in the complementary treatment of immunocompromised patients (e.g., patients with AIDS, HIV infection and transplants). With respect to bronchial asthma, for example, the compositions of the present invention increase the production of PGE₁ which reduces inflammation by increasing the level of cAMP, thus inhibiting the release of A A′ from stores and activating T-lymphocytes.

The dosage regimen for treating the diseases described herein is selected in accordance with a variety of factors, including the age, weight, sex, and medical condition of the patient, the severity of the disease, the route of administration, pharmacological considerations such as the activity, efficacy, pharmacokinetic and toxicology profiles of the particular compound or composition used, whether a drug delivery system is used and whether the compound or composition is administered as part of a drug combination.

The compounds and compositions of the present invention can be administered topically, orally, parenterally, by inhalation (nasal or oral), vaginally, or rectally in dosage unit formulations containing conventional nontoxic pharmaceutically acceptable carriers, adjuvants, and vehicles, as desired. One skilled in the art will appreciate that the compounds described herein may be present in the form of various conventional pharmaceutically acceptable salts. The term parenteral as used herein includes subcutaneous, intravenous, intramuscular, intrasternal injection, or infusion techniques. Preferably, the compounds or compositions of the present invention are topically administered.

Injectable preparations, for example, sterile injectable aqueous or oleaginous suspensions may be formulated according to the known art using suitable dispersing or wetting agents, suspending agents (e.g., methylcellulose, Polysorbate 80, hydroxyethylcellulose, sodium carboxymethylcellulose, polyoxytehylene sorbitan monolaurate and the like), pH modifiers, buffers, solubilizing agents (e.g., polyoxyethylene hydrogenated castor oil, Polysorbate 80, nicotinamide, polyoxyethylene sorbitan monolaurate, Macrogol, an ethyl ester of castor oil fatty acid, and the like) and preservatives. The sterile injectable preparation may also be a sterile injectable solution or suspension in a nontoxic parenterally acceptable diluent or solvent, for example, as a solution in 1,3-butanediol. Among the acceptable vehicles and solvents that may be used are water, Ringer's solution, and isotonic sodium chloride solution. In addition, sterile, total lipids are conventionally used as a solvent or suspending medium. For this purpose any bland total lipid may be used including synthetic mono- or diglycerides, in addition, fatty acids such as oleic acid find use in the preparation of injectables. The preparations can be lyophilized by methods known in the art.

Solid dosage forms for oral administration may include capsules, soft gel capsules, tablets, sublingual tablets, powders, and granules. In such solid dosage forms, the active compound(s) may be admixed with one or more inert diluents such as lactose or starch. As is normal practice, such dosage forms may also comprise other substances including lubricating agents such as magnesium stearate. In the case of capsules, tablets, and pills, the dosage forms may also comprise buffering agents. The tablets can be prepared with enteric or film coatings. For treating bronchial asthma in adults, the compositions of the present invention are preferably prepared in the form of a soft gel capsule.

Liquid dosage forms for oral administration can include pharmaceutically acceptable emulsions, solutions, suspensions, and syrups containing inert diluents commonly used in the art, such as water. Such compositions can also comprise adjuvants, such as wetting agents, emulsifying and suspending agents, and sweetening, flavoring, and perfuming agents. When pharmaceutical preparations of the present invention are prepared for treating infants or children, they are preferably prepared in a liquid dosage form. For example, in the methods of treating bronchial asthma described herein, the composition is preferably prepared in the form of a syrup when it is administered to infants or children.

For administration by inhalation (oral or nasal), the compositions of the invention can be delivered from an insufflator, a nebulizer or a pressured pack or other convenient mode of delivering an aerosol spray. Pressurized packs can include a suitable propellant. Alternatively, for administration by inhalation, the compositions can be administered in the form of a dry powder composition or in the form of a liquid spray.

Suppositories for vaginal, urethral, or rectal administration can be prepared by mixing the active compounds with suitable nonirritating excipients such as cocoa butter and polyethylene glycols that are solid at room temperature and melt at body temperature.

For topical administration, the compounds or compositions of the invention can be formulated, for example, as an ointment, cream, gel, powder, balm (e.g., lip balm, stick deodorant), or lotion, or as the active ingredient of a transdermal patch. Topical administration can also be accomplished with a liquid spray, an aerosol, or via iontophoresis, or through the use of liposomes, microbubbles or microcapsules. Ointments and creams may be formulated, for example, with an aqueous or oily base with the addition of suitable thickening (e.g., PEG 4000, PEG 6000, wax, hard paraffin) and/or gelling agents (e.g., hydroxypropyl cellulose). Lotions may be formulated with an aqueous or oily base and can also generally contain one or more emulsifying agents (e.g., wool wax alcohol, fatty acid glycol esters), stabilizing agents (e.g., polyoxyethylene sorbitan monolaurate, carboxy methyl cellulose), dispersing agents (e.g., sodium oleate, propylene glycol), suspending agents (e.g., methyl cellulose, chitosan, accacia, carboxymethyl cellulose, tragacanth, pectin), thickening agents, and/or coloring agents (e.g., dyes, lackes). Topical administration includes administration to the vulva and vagina.

Generally, a lotion is a suspension of finely divided active ingredient in a small amount of water. Lotions provide fast release of the active ingredient due to the soluble nature of the active ingredient and the water content. Lotions generally have short duration of action. Generally, an ointment is a semi-solid preparation that is more viscous and provides for slow release of the active ingredients due to longer duration of contact with the skin. Generally, a cream is a semi-solid preparation that contains a humectant and a higher percentage of water than an ointment. Creams are less viscous than ointments and provide for release of the active ingredients over a moderate period of time.

In preferred embodiments, the composition is in the form of a topical composition (e.g., ointment, cream, gel., or the like), in the form of a suppository, or in a liquid dosage formulation (e.g., syrup). In another preferred embodiment, the present invention provides a novel composition in the form of a liquid dosage formulation (e.g., a syrup) containing the Nigella sativa L. lipid fraction described herein in an amount of about 5% to about 15% by weight, more preferably in an amount of about 9 to about 11% by weight, most preferably in an amount of about 10% by weight, based on 100% by weight of the composition.

While the compounds of the invention can be administered as the sole active pharmaceutical agent in the methods described herein, they can also be used in combination with one or more compounds which are known to be therapeutically effective against the specific disease that one is targeting for treatment.

The present invention is described in terms of a Nigella sativa L. lipid fraction. The methods for obtaining a Nigella sativa L. lipid fraction can also be followed to obtain a Nigella arvensis lipid fraction, a Nigella damascena lipid fraction, or a lipid fraction from any other species of Nigella. Moreover, a Nigella arvensis lipid fraction and/or a Nigella damascena lipid fraction can be used for treating wounds, treating skin and soft tissue infections caused by gram-positive organisms or gram-negative bacilli, modulating bacterial growth, treating and preventing bacterial infections, treating pyogenic skin infections, treating cellulite, regulating and normalizing cardiovascular parameters, treating nipple fissures, treating septic infections, and treating and preventing respiratory diseases and disorders, as described herein.

### EXAMPLES

The following examples are for purposes of illustration only, and are not intended to limit the scope of the appended claims.

### Example 1

A process for preparing the Nigella sativa L. lipid fraction of the present invention is outlined in FIG. 1 and is described below.

4 kilograms of crushed Nigella sativa L. seeds were successively extracted in a percolator until exhaustion with various solvents in order of increasing polarity. The solvents used, in order, were petroleum ether (with boiling point between 40-60° C.) or hexane, ether, chloroform, ethylacetate, acetone, ethanol, methanol and water.

A slurry composed of 1 part silica gel G. and 2 parts water was spread on clean glass plates at a thickness of 0.25-0.3 mm. The plates were air-dried for 30 minutes, activated in an air-drying oven at 110° C. for 30 minutes, and then kept in a dissector until use.

5.0 grams of the intermediate product (e.g., the petroleum ether extract) prepared by extraction were dissolved in petroleum ether and mixed with about 5 grams of silica gel 60 (For Column) to form a homogenous mixture, were finely powdered, and applied on the top of a column (3×60 cm) packed with silica gel 60 (250 grams). Elution was performed using the best solvent system as indicated by TLC, in this case ether:benzene (85:15). Fractions of 50 ml were collected and the similar fractions, as indicated by TLC, using different solvent systems, preferably ether:benzene (85:15), and vanillin H₂SO₄ spray reagent, were pooled together and then evaporated under reduced pressure to yield the Nigella sativa L. total lipid fraction of the present invention.

### Example 2

The following pharmaceutical formulation was prepared in the form of a liquid syrup. Each 100 ml of syrup contained the following: Active Ingredient (10 ml); Gum acacia (4 ml); Flavor (0.05 ml); and simple syrup (66.7% sucrose in water) added to make 100 ml total. The “active ingredient” refers to the Nigella sativa L. lipid fraction of the present invention.

### Example 3

The following pharmaceutical formulation was prepared in the form of a soft gel capsule. Each 715 mg soft gel capsule contained 500 mg (ranging from about 490 to about 510 mg) of the Nigella sativa L. lipid fraction of the present invention (e.g., 424 mg total fatty acids (which contains 344.4 mg polyunsaturated fatty acids, 65.6 mg saturated fatty acids, 2.5 mg volatile oils, 11.5 mg sterols, and 76 mg other components, such as short chain fatty acids, waxes, resins and hydrocarbons). The soft gel capsule shell comprised 18% gelatin, 10% glycerin, 0.6% titanium dioxide, 2% purified water, and 0.8% color. The soft gel capsule shell weighed about 220 to about 240 mg.

### Example 4

The following pharmaceutical formula was prepared in the form of a cream. Each 100 grams contained 5% (i.e., 5 grams) of the Nigella sativa L. lipid fraction as the active ingredient. The inactive ingredients were a cream base containing cetyl alcohol, glyceryl monostearate, Span 80, purified water, Tween 80, methyl cellulose, propylene glycol, BHA, BHT, methylparaben and propylparaben.

### Example 5

### In Vitro Antimicrobial Comparison Study

The comparative antibacterial activity of ampicillin (1 microg/mg) and the Nigella sativa L. lipid fraction (1 mg/ml) against Staphylococcus pyogenes, Corynebacterium pyogenes and Salmonella typhi murium was performed in vitro using the bore method described by Cooper and Woodman (1964).

The results shown in the table below (data are the mean ±S.E. of 4 observations) indicate that the antibacterial effect of the Nigella sativa L. lipid fraction (1 mg/ml) was almost equipotent to ampicillin (1: g/mg) against Staphylococcus pyogenes, Corynebacterium pyogenes and Salmonella typhimurium.

TABLE 3

| | Diameter of Zone of inhibition (mm) | |
|---|---|---|
| | | Nigella sativa L. |
| | | lipid fraction |
| Strain of bacteria | Ampicillin 1: g/mg | (1 mg/ml) |
| Staphylococcus pyogenes | 26.7 ± 0.3 | 24.9 ± 0.3 |
| Corynebacterium pyogenes | 44.7 ± 0.8 | 44.6 ± 0.7 |
| Salmonella typhi murium | 13.1 ± 0.2 | 14.0 ± 0.0 |

### Example 6

### In Vivo Experimental studies

The effects of the Nigella sativa L. lipid fraction on septic wound healing was evaluated on experimentally-induced septic wounds in guinea pigs. Forty male and female guinea pigs (400-600 g) were divided into 4 groups, 10 animals/group. The first group was a negative control group that was treated by saline dressings. The second group was a positive control group treated by acriflavine (1%)—teramycin dressings. Acriflavine is a natural dye used as for preoperative skin sterilization and as a cream for wounds and burns. The third group was treated by the Nigella sativa L. lipid fraction (5%) dressings. The fourth group was treated by the Nigella sativa L. lipid fraction (1%) dressing.

A 2 cm length of surgical incision was induced in each animal. Wounds were infected with a 106 inoculum of Staphylococcus pyogenes and animals were left for the following 2 days to ensure sepsis. Infected wounds were then dreaded by topical dressings of different medication according to the protocol.

Evaluation of wound healing was assessed by daily measurement of the wound length and complete healing was indicated by complete closure of the wound and growth of hair. Bacterial cultures were done using swabs from wounds to demonstrate the eradication of bacteria (Staphylococcus pyogenes).

The results reported in the table below indicate that the wounds dressed with saline (control group) healed completely after 15.8 days while wounds dressed with Acriflavine-teramycin healed after 10.6 days. Wounds treated with the Nigella sativa L. lipid fraction (1 and 5%) dressings showed complete healing after 9.6 and 9.2 days, respectively.

Significant differences in the rate of wound healing (P<0.05) were observed between control wound dressed by saline versus wounds dressed by either Acriflavine-teramycin and the Nigella sativa L. lipid fraction (1% and 5%). No statistical difference in the rate of wound healing was observed between wound dressed by Acriflavine-teramycin and the Nigella sativa L. lipid fraction (1% and 5%). Bacterial swab cultures confirmed the absence of Staphylococcus pyogenes growth.

TABLE 4

| | 5 Days | | 7 Days | | 9 days | | |
|---|---|---|---|---|---|---|---|
| | Wound | Healing | Wound | Healing | Wound | Healing | Days for |
| | length | degree | length | degree | length | degree | complete |
| Group | (mm) | (mm) | (mm) | (mm) | (mm) | (mm) | healing |
| Control | 4.50 ± 0.20 | 0.40 ± 0.24 | 3.74 ± 0.11 | 1.20 ± 0.20 | 2.00 ± 0.12 | 2.5 ± 0.09 | 14.6 ± 0.50 |
| Acriflavine- | 3.18** ± 0.13 | 0.80 ± 0.37 | 2.80** ± 0.25 | 2.40** ± 0.24 | 0.90** ± 0.08 | 3.20** ± 0.24 | 10.6 ± 0.37** |
| terramycin | | | | | | | |
| Nigella sativa | 2.68** ± 0.25 | 1.20** ± 0.17 | 1.94** ± 0.54 | 2.60* ± 0.40 | 0.30** ± 0.02 | 3.60** ± 0.40 | 9.6 ± 0.24** |
| L. lipid | | | | | | | |
| fraction (1%) | | | | | | | |
| Nigella sativa | 1.80** ± 0.31 | 1.80** ± 0.27 | 1.50** ± 0.31 | 3.00** ± 0.00 | 0.10** ± 0.00 | 3.90** ± 0.10 | 9.2 ± 0.20** |
| L. lipid | | | | | | | |
| fraction (5%) | | | | | | | |

In the table above, the values are the mean ± S.E. of observations from 5 guinea pigs.
*Statistically significant from negative control at P < 0.05.
**Statistically significant from negative control at P < 0.01).

### Example 7

### Phase I Clinical Trials

Ten healthy male volunteers subjects were selected for this study. All patients were screened according to their medical history, physical and laboratory investigations e.g. chest, x-ray ECG, liver and kidney functions tests and also skin examination. Patients were excluded from the study if they were diabetics, had hepatic or renal diseases complained from skin lesions, or neurological disorders. Subjects with emotional or psychological instability were excluded. Patients willing to participate in the study had a complete physical and skin examination in addition to laboratory investigations.

Subjects were asked to apply the cream described in Example 4 (about 0.5 grams twice daily for two weeks) topically into an area (about 5 cm in diameter) on the medical aspect of the skin of the left arm. They were hospitalized for 48 hours where all vital signs were monitored and laboratory investigations (including liver and kidney function tests, hematological studies, urine and stool analysis in addition to an ECG and a chests x-ray) were done immediately before and by the end of the period of application of the cream of Example 4. Subjects were instructed to attend the outpatient clinic of the hospital once weekly for a check on the 14th day, they were readmitted for clinical and laboratory evaluation. Prior to the trial signed consents were obtained from the volunteers.

Results of the study demonstrate no local irritation or sensitization observed in the subjects. Physical examination and laboratory investigations carried out at the end of the fourth week revealed no statistically different changes. The local preparation of the cream described in Example 4 was well tolerated in all the subjects enrolled in this trial without any observable local or systemic side effects.

### Example 8

### Phase II Clinical Trials

The aim of this study was to study the effectiveness of the topical cream in Example 4 in the treatment of infected wound through an open randomized trial. 84 patients with infected wounds were selected from the outpatient clinics of some hospitals in Cairo, Egypt. Patients were chosen according to clinical diagnosis. All patients were screened according to their medical and laboratory investigations and physical examination, e.g., chest x-ray, ECG, liver and kidney function tests. Urine and stool analysis was performed. Patients were excluded from the study if they were diabetics, had emotional or psychological instability or were taking immunosuppressive agents such as cortisone.

Patients recruited for the trial were selected if they had pyogenic skin infections of one of the following types: impetigo, folliculitis, infected eczema, infected wounds, flea bites, leg ulcers, and erythema. Male and female patients with ages ranging from childhood to elderly were subjected to a complete physical examination. They were enrolled in the study for two weeks and given the composition in Example 4 in the form of an ointment, cream or lotion according to their lesions. They were asked to apply it twice daily (morning and evening) for two weeks. Patients' urine was tested for glucosuria. Patients involved in the study were followed up twice weekly. Prior to the trial and a signed consent was obtained.

Results of the study which included 78 patients having pyoderma, impetigo, folliculitis or secondary infections as infected wounds, eczema and fleabites indicated that the use of the composition in Example 4 achieved a 77% success (73% cure and 4% improvement). In other lesions (i.e., leg ulcers and erythema), 6 cases achieved 83% success (50% cure and 33% improvement).

These results were similar to those observed using conventional preparations as shown in the table below.

TABLE 5

| | | Complete Cure | | Improvement | | No Response | |
|---|---|---|---|---|---|---|---|
| | # of | # of | % | # of | % | # of | % |
| Type of Infection | patients | patients | response | patients | response | patients | response |
| Pyoderma (impetigo, | 78 | 57 | 73 | 3 | 4 | 18 | 23 |
| folliculitis, and | | | | | | | |
| secondary infections, | | | | | | | |
| such as infected | | | | | | | |
| eczema, infected | | | | | | | |
| wounds, flea bites) | | | | | | | |
| leg ulcers and erythema | 6 | 3 | 50 | 2 | 33 | 1 | 17 |
| | 84 | 60 | 71 | 5 | 6 | 19 | 23 |

Complete cure was identified as a clinical and bacteriological cure (scraping and culture). Improvement was identified as a clinical cure (reduction in the size of the original lesion without complete disappearance). No response was when neither clinical nor bacteriological improvement occurred.

In summary, the composition described in Example 4 is effective in the treatment of pyogenic skin infections and as a topical antibacterial preparation.

### Example 9

The Nigella sativa L. lipid fraction of the present invention was formulated as an orally administrable syrup (particularly for infants and children) composed of a simple syrup with 10% active ingredients (i.e., the Nigella sativa L. lipid fraction of the present invention), 66.7% sucrose in water and gum acacia (e.g., about 4 grams) as an emulsifying agent.

### Example 10

The Nigella sativa L. lipid fraction of the present invention was formulated as an orally administrable soft gelatin capsule composed of 10% active ingredients (i.e., the Nigella sativa L. lipid fraction of the present invention), 66.7% sucrose in water, and gum acacia (e.g., about 4 grams) as an emulsifying agent.

### Example 11

This example explains in vivo studies to evaluate the prophylactic (protective) anti-asthmatic effect of the Nigella sativa L. lipid fraction of the present invention. The prophylactic anti-asthmatic effect was evaluated in vivo by measurement of the preconvulsive time in guinea pigs exposed to histamine aerosol (0.25%).

Guinea pigs were divided into three groups with 8 animals per group. The first group received saline with injection to serve as negative control. The second group received ketotifen, which is the standard treatment to serve as positive control. The third group received 200 mg/kg each of the Nigella sativa L. lipid fraction of the present invention.

Animals in all groups were treated for 21 days following, after which they were exposed to histamine (0.25%) aerosol and the preconvulsive time was recorded. The results are shown in the table below.

TABLE 6

| | | % of | | |
|---|---|---|---|---|
| | Preconvulsive time | Prophylaxis | | |
| Treatment | mean ± SD (seconds) | (Protection) | Dosage | |
| saline - negative | 71.6 ± 14.6 | 11.9 | | |
| control | | | | |
| ketotifen | 497.1* ± 183.2 | 82.85 | 50: | g/kg |
| positive standard | | | | |
| Nigella sativa L. | 312.6* ± 162.9 | 52 | 200 | mg/kg |
| lipid fraction | | | | |

*Statistically significant from the control group (P < 0.05).

### Example 12

This experiment was an in vivo study to evaluate the tachypnea (protective) effect of the Nigella sativa L. lipid fraction of the present invention. The second protocol involved sensitized animals. Animals were sensitized by injecting ova albumin in two doses 100 mg each (S.C. and I.P., simultaneously). Animals were resensitized 15 days later. Guinea pigs were divided into three groups with 8 animals per group. The first group received a saline injection to serve as negative control. The second group received ketotifen which is the standard treatment to serve as positive control. The third group received 200 mg/kg each of the Nigella sativa L. lipid fraction of the present invention.

Animals in all groups were treated for 21 days following which they were exposed to ova albumin inhalation and time to onset of tachypnea was recorded as shown in the table below.

TABLE 7

| | Time to attain | | | |
|---|---|---|---|---|
| | Tachypnea | | | |
| | mean ± SD | % of | | |
| Treatment | (seconds) | Prophylaxis | Dosage | |
| saline - negative | 81.4 ± 33.3 | 13.6 | | |
| control | | | | |
| ketotifen | 266.3* ± 128 | 44.4 | 50: | g/kg |
| positive standard | | | | |
| Nigella sativa L. | 206.7* ± 97.4 | 34.4 | 200 | mg/kg |
| lipid fraction | | | | |

*Statistically significant from the control group (P < 0.05).

### Example 13

### Clinical Trials

The objective of the clinical trials was to study the effectiveness of the Nigella sativa L. lipid fraction of the present invention in the form of a syrup and soft gelatin capsule in the treatment and prophylaxis of bronchial asthma in children and adult patients.

52 patients (34 children and 18 adults) with bronchial asthma were selected from outpatient clinics of some hospitals in Cairo, Egypt. Patients were chosen according to the clinical and laboratory diagnosis. Patients were screened according to their medical history. A physical examination and laboratory investigations for HB %, CBC, liver function tests, and kidney function tests were performed. Patients were excluded from the study if there were diabetics, had hepatic pulmonary or renal diseases, or had infections or chronic ulcers. Patients suffering from thyortoxicosis, cardiac problems, taking steroid medications, C.N.S. depressants medications and/or immunosuppressive agents, such as corticosteroids were excluded. Patients willing to participate in the study had to complete a physical examination, laboratory investigations as described above and a chest X-ray examination.

Patients enrolled in the study were given the Nigella sativa L. lipid fraction of the present invention (20 mg/kg/day) in the form of 4 capsules of 400 mg each for adults weighing 80 Kg (Example 10) or syrup for children (Example 9). The patients did not receive any other medications.

Patients were advised to exercise caution to avoid exposure to an acute attack of obstructive lung disease. At the beginning of the trial ECG examination was performed to all adult participants. A respirometer and a peak flow meter were used to measure vital lung capacity in children, particularly expiratory volume. Patients were instructed to report twice weekly to the medical center to assess the lung capacity. Patients were instructed to come immediately to the center if complaining from any chest problems during the study.

The results of the study demonstrate marked protection from attacks of bronchial asthma in both children and adults. Frequency rates were decreased from 1.5 and 4 to 0.27 and 0.43 attacks/month in children and adults, respectively. Expiratory volume in children significantly increased indicating improvement of lung function as well.

The effect of the Nigella sativa L. lipid fraction of the present invention on the prophylaxis from bronchial asthma in children and adults is shown in the table below.

TABLE 8

| | | Frequency of | | | |
|---|---|---|---|---|---|
| | | attacks/month | | Expiratory volume (ml) | |
| Bronchial | Treatment | Before | After | Before | After |
| asthma patients | regimen | treatment | treatment | treatment | treatment |
| Children (34) | Example 9 | 1.5 ± 0.7 | 0.27 ± 0.24 | 167 ± 67 | 216 ± 45 |
| | 20 mg/kg/day | | | | |
| | In two divided | | | | |
| | doses | | | | |
| Adults (18) | Example 10 | 4 | 0.43 | Not Done | Not Done |
| | 2 capsules | | | | |
| | (400 mg each) | | | | |
| | twice daily | | | | |

All publications, including, but not limited to, patents and patent applications cited in this specification, are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the pharmaceutical art can, given the preceding description, utilize the present invention to its fullest extent, using no more than routine experimentation. Therefore any examples are to be construed as merely illustrative and not a limitation on the scope of the present invention in any way. Accordingly, it will be apparent to one skilled in the art that various modifications can be made to the invention without departing from the spirit or scope of the appended claims.

1. (canceled)

2. (canceled)

3. (canceled)

4. (canceled)

5. (canceled)

6. A method of treating a pyogenic skin infection in a patient in need thereof comprising topically administering an effective amount of a topical composition comprising a lipid fraction extracted from Nigella sativa L. seeds and a pharmaceutically acceptable carrier.

7. The method of claim 6, wherein the pyogenic skin infection is selected from the group consisting of pyoderma, impetigo, folliculitis, eczema, an infected wound, an insect bite, a dermal injury, a leg ulcer and erythema.

8. A method of treating a bacterial infection in a patient in need thereof comprising topically administering an effective amount of a topical composition comprising a lipid fraction extracted from Nigella sativa L. seeds and a pharmaceutically acceptable carrier.

9. A method for healing a wound in a patient in need thereof comprising topically administering an effective amount of a topical composition comprising a lipid fraction extracted from Nigella sativa L. seeds and a pharmaceutically acceptable carrier.

10. The method of claim 9, wherein the wound is a diabetic leg ulcer.

11. A method for treating skin and soft tissue infections caused by gram-positive organisms or gram-negative bacilli in a patient in need thereof comprising topically administering an effective amount of a topical composition comprising a lipid fraction extracted from Nigella sativa L. seeds and a pharmaceutically acceptable carrier.

12. A method for treating cellulite in a patient in need thereof comprising topically administering an effective amount of a topical composition comprising a lipid fraction extracted from Nigella sativa L. seeds and a pharmaceutically acceptable carrier.

13. A method for treating a septic infection in a patient in need thereof comprising topically administering an effective amount of a topical composition comprising a lipid fraction extracted from Nigella sativa L. seeds and a pharmaceutically acceptable carrier.

14. A method of treating or preventing a vaginal disease or disorder in a patient in need thereof comprising topically administering an effective amount of a topical composition comprising a lipid fraction extracted from Nigella sativa L. seeds and a pharmaceutically acceptable carrier.

15. The method of claim 14, wherein the vaginal disease or disorder is selected from the group consisting of vaginal moniliasis, vaginitis, vaginal discharge, vaginal itching, vaginal inflammation, candidiasis, vulvovaginitis, bacterial vaginitis, trichmonas vaginitis, cervicitis, vaginal irritation, pruritis, vaginal burning, atrophic vaginitis and a sexual transmitted disease.

16. A method for treating or preventing vaginal moniliasis in an immuno-compromised patient in need thereof comprising topically administering an effective amount of a topical composition comprising a lipid fraction extracted from Nigella saliva L. seeds and a pharmaceutically acceptable carrier.

17. A method of treating a respiratory disease or disorder in a patient in need thereof comprising topically administering an effective amount of a topical composition comprising a lipid fraction extracted from Nigella sativa L. seeds and a pharmaceutically acceptable carrier.

18. The method of claim 17, wherein the respiratory disease or disorder is selected from the group consisting of bronchospasm, bronchoconstriction, anaphylaxis, pulmonary tuberculosis, an inflamed lung tissue, an inflamed mucous membrane, asthma, a respiratory tract inflammation, bronchitis, bronchial hyperactivity, a dry cough, bronchial congestion, a throat irritation, and an upper respiratory allergy.

19. The method of claim 17, wherein the topical composition comprises an evaporative chest rub.
